(19) United States
(12) Patent Application Publication
(10) Pub. No.: US 2020/0399330 A1
(43) Pub. Date: Dec. 24, 2020

(54) ANTI-LEUKEMIC, ANTI-HIV, AND SIALIDASE ACTIVITIES OF ROYAL-JELLY PROTEINS

(71) Applicants: Salem Ismaell Salem EL-FIKY, Alexandria (EG); Marwa Muhammad Abu-Serie ALI, Alexandria (EG); Noha Hassan Habashy Mohammad ABD AL-GALIL, Alexandria (EG)

(72) Inventors: Salem Ismaell Salem EL-FIKY, Alexandria (EG); Marwa Muhammad Abu-Serie ALI, Alexandria (EG); Noha Hassan Habashy Mohammad ABD AL-GALIL, Alexandria (EG)

(21) Appl. No.: 16/961,667

(22) PCT Filed: Sep. 6, 2018
(86) PCT No.: PCT/EG18/00012

§ 371 Date: Jul. 11, 2020

(51) Int. Cl. C07K 14/435; A61K 35/644; A61P 35/00; A61P 31/18

(52) U.S. Cl. CPC C07K 14/43572; A61K 35/644; A61K 38/00; A61P 31/18; A61P 35/00

(57) ABSTRACT

This invention discloses isolated protein fractions from Apis mellifera royal jelly (RJ) have proven potent efficacy in inhibiting leukemia cell growth and HIV-1 reverse transcriptase (RT) as well as releasing the cellular sialic acid (sialidase catalytic activity). Methods for RJ fractionation, the investigation against leukemia cell lines (NFS-60 and Jurkat cells), HIV-1 RT, and cellular SA are disclosed.

### TECHNICAL FIELD

The present invention relates to novel natural proteins having anti-leukemic, anti-human immunodeficiency virus (HIV), and sialidase activities. In particular, the current invention relates to protein fractions purified from Apis mellifera royal jelly (RJ) having high cytotoxic activity against leukemia cells, inhibit HIV-1 transcriptase activity, and release sialic acid from the PBMCs and HepG2 cells.

### BACKGROUND ART

Societies face many problems that harm their economy and their citizens and therefore strive to solve them by all possible means to provide a better life. The most important of these problems is the spread of incurable diseases that have no treatment or have unsafe treatments without available alternatives. Infections with viruses, some bacteria, and cancer are among the most important diseases that the whole world faces.

Leukemia is a type of cancer in which certain white blood cells grow out of control and spread through the bloodstream. It can be broadly classified into lymphocytic leukemia and myelogenous leukemia depending on the type of cell lineage affected. There were 352, 000 new cases and 265, 000 deaths of leukemia estimated worldwide in 2012 and an estimation of more than 20 million new cases will appear for 2025. The treatment involved chemotherapy, radiation therapy, or stem cell transplantation. However, these treatments have various side effects, so seeking for a new alternative safe therapy is the need of the day.

Viral infection is one of the biggest problems that facing human in his life. Viruses can enter the human cells through specific receptors such as sialic acid (SA). The SA generally binds terminally to different cell surfaces and secreted glycoconjugates monosaccharides. It is involved in vital interactions between cells and various viruses as well as other pathogens at many points in their infection and transmission cycles. It may act as a primary receptor for cell infection, or a component in a series of interactions that lead to infection. Some examples of viruses that depend on SA in their infection, including Influenza, Isavirus, Coronaviruses, Respirovirus, Rubulavirus, Avulavirus, Adenoviruses, and others. Beside some gram-positive and negative bacteria and their toxins such as Helicobacter pylori, Streptococcus pneumoniae, Vibrio choleraetoxin, Clostridium tetanitoxin, and others. Mucosal surfaces are further protected by secretion of mucus in which SA acts through binding and trapping viruses and prevent them from accessing their target tissues and remove them through a process mediated by mucociliary transport.

Viruses replicated in many different ways, some of them depend on a type of enzyme called reverse transcriptase, which converts RNA into DNA to enable it to bind to the cellular DNA forming its important proteins. These types of viruses are characterized by their inclusion of RNA so they called Retroviruses and fall under a large family called (Retroviridae). This family includes seven different viral strains, the most important of which are HIV, and Oncoviruses, which affect the immune system, nervous system, etc., leading to AIDS, autoimmune disease, cancer, and various neurological diseases. Also, hepatitis B virus (HBV) which belongs to the Hepadnaviridae family contains the RT. The currently approved therapy for these viruses is reverse transcriptase and protease inhibitors and most of which are in the form of nucleoside analogs. All of these treatments caused many side effects.

RJ (bee's milk) is a creamy, whitish product secreted from mandibular and hypopharyngeal glands of nurse bees (Apis mellifera). It is the specific food for the queen bees and helps in their development from the worker bee larva with the age of 5-15 days. RT is a highly acidic substance composed mainly of water (60-70%), proteins (9-18%), sugars (7-18%), and lipids (3-8%) with other compounds. About 80% of the RJ proteins are water-soluble belonging to the major RJ protein (MRJP) family, which comprises 9 members (MRJP1-MRJP9) with a molecular mass of 49-87 kDa. To date, the anti-leukemic, anti-HIV and the ability of RJ and its components to release the cellular SA are undefined.

### SUMMARY OF THE INVENTION

RJ (FIG. 1) was fractionated as elucidated in our recently published PCT (EG2017/000022) into carbohydrate, lipid, and crude protein fractions. Then the crude protein fraction (CPF) was further fractionated into five fractions (PF₂₅, PF₃₀, PF₄₀, PF₅₀, and PF₆₀) using different ammonium sulfate saturation [(20-25), (25-30), (30-40), (40-50) and (50-60%), respectively]. Also, PF₅₀ was further fractionated into two purified proteins, major RT protein 2 (MRJP2) and its isoform X1. The lyophilized fractions of RT were tested against murine myeloid and humanT lymphocyte leukemia cell lines (NFS-60 and Jurkat cells, respectively) and the activity of the HIV-1 reverse transcriptase using published methodology. In addition, the seven protein fractions were tested for their ability to remove the cellular SA (sialidase activity) using two types of cells, peripheral blood mononuclear cells (PBMCs) and HepG2 cells.

The PF₅₀ and MRJP2 exhibited the most effective RT fraction against the two types of leukemia cells by inducing apoptosis and by almost completely blocking the cell cycle G0 phase within 72 h. In addition, one milligram of each PF₂₅, PF₃₀, PF₄₀, PF₆₀, or MRJP2 inhibited the HIV-1 RT by more than 90%. On the other hand, the five protein fractions (PF₃₀, PF₄₀, PF₅₀, MRJP2, MRJP2 X1) have proven their sialidase activity after 2 h and 72 h incubation with the PBMCs and HepG2 cells and PF₅₀ was the most effective.

As used herein, the term “chemotherapy” refers to the treatment of disease by means of chemicals and it is the main drug therapy for any type of cancer such as leukemia.

The term “stem-cell” means is a type of cell that can produce other cells that are able to develop into any kind of cell in the body. The term “stem cell transplantation” as used herein refers to a procedure that replaces unhealthy blood-forming cells with healthy cells. It is an option for the treatment of leukemic patients.

The term “SA” means any N-acyl derivative of neuraminic acid. Various ones are found in polysaccharides, glycoproteins, and glycolipids. SA is a group of amino carbohydrates presents in cell membrane attached to the monosaccharides and perform various functions such as binding to various types of viruses, bacteria, and toxins to facilitate their actions in the occurrence of the disease.

The term “receptor” refers to a molecule on the cell surface (cell-surface or membrane receptor) or within a cell, usually in its nucleus (nuclear receptor) that recognizes and binds with specific molecules, producing certain effects in the cell. As herein SA act as a membrane receptor for many viruses, they bind with it to penetrate the cell and begin their lifecycle.

The term “gram-bacteria” is a term used by a microbiologist to classify bacteria into two groups (gram-positive or gram-negative) based on the bacterium's chemical and physical cell wall properties. Gram-positive bacteria are referred to monoderms having one membrane, and gram-negative bacteria are referred to as diderms, having two membranes. As herein, some gram-positive and gram-negative bacteria can bind to the cellular SA to initiate their infection.

The term “analog” means similar in some way. As herein, nucleoside analogs refer to a structural analog of a nucleoside, a category that includes both purine analogs (like the antiviral agents) and pyrimidine analogs (like the anticancer agents).

The term “fractionation” means a separation process in which a certain mixture is divided into a number of smaller quantities (fractions). The term “protein purification” as used herein refers to a technique by which a single protein type is isolated from a complex mixture. Therefore, the protein fraction may contain two (For example, PF₃₀ and PF₅₀) or more (For example, PF₆₀ and the CPF) proteins. The purified protein or fraction means single protein with fewer impurities (For example MRJP2 and its isoform X1).

The term “isoform” as used herein means a protein that has the same function as the original protein but which is encoded by a different gene and may have small differences in its sequence. Here the MRJP2 and its isoform X1 are encoded by different genes and having slightly different in their sequences.

The term “hepatocytes” refers to the epithelial parenchymatous cells of the liver which make up 70-85% of the liver's mass and responsible for most of the liver functions.

The term “lyophilized” or “freeze-dried” means to dry something (For example, food) in a frozen state under high vacuum especially for preservation. In this invention, the isolated fractions from RJ were freeze-dried to obtain the powdered form for accurate preparation of different concentrations for the analyses.

As used herein, the term “crude-protein” means all the water-soluble proteins in the RJ.

### DETAILED DESCRIPTION

This invention provides certain protein fractions from RJ (obtained from the local market, Egypt) having high cytotoxic potency against leukemia cell growth, inhibitors for HIV-1 replication, and able to release the cellular SA (sialidase catalytic activity).

### RJ Fractionation

RJ was fractionated following the method that used in our recently published PCT (EG2017/000022) into carbohydrate, lipid, and protein fractions.

Carbohydrate fraction preparation, 2 g of RJ was dissolved in water/methanol mixture (3:1) and deproteinized using Carrez I (potassium hexacyanoferrate II) and Carrez II (zinc acetate) reagents. Then, lipids were removed by washing the deproteinized RJ two times with dichloromethane. The aqueous layer (sugar fraction) was filtered through 0.2 μm disposable syringe filter, lyophilized (Telstar, Terrassa, Spain) and kept at −80° C. until used.

Lipid fraction preparation, lipids were isolated from RJ with petroleum ether using Soxhlet apparatus for 30 min. The organic solvent was evaporated, and then the lipid fraction was stored at −80° C.

Crude protein fraction (CPF) preparation, the water-soluble proteins were extracted from RJ using ammonium sulfate crystals (Brixworth, Northants, UK). In brief, 1.5 g of RJ was dissolved in phosphate buffer saline (PBS, 0.1 M, pH 7) containing 1× protease inhibitor cocktail (Sigma-Aldrich, St. Louis, Mo., USA) and the solution was centrifuged at 3800 g and 4° C. for 30 min. Then the water-soluble proteins in the supernatant were precipitated by adding crystals of ammonium sulfate until the saturation reach 60%. Pellet (CPF) was dissolved in PBS, dialyzed for 24 h against the same buffer and finally freeze-dried to obtain the powdered fraction.

The RJ CPF fractionation, CPF was further fractionated into five fractions (PF₂₅, PF₃₀, PF₄₀, PF₅₀, and PF₆₀) using different ammonium sulfate saturation (20-25%, 25-30%, 30-40%, 40-50%, and 50-60%, respectively). The precipitated proteins were obtained by centrifugation at 3800 g (4° C.) for 30 min, dialyzed for 24 h against PBS, and lyophilized.

The major RJ protein 2 (MRJP2) and its isoform X1 purification, The PF₅₀ was further fractionated by carboxymethyl (CM)-Sephadex ion-exchange column chromatography into two purified proteins, major RJ protein 2 (MRJP2) and its isoform X1. In brief, the amount of PF₅₀ that obtained from 10 g of RJ was dissolved in 20 mL of the binding buffer (20 mM phosphate buffer containing 1× protease inhibitor cocktail, pH 6.7). The protein solution then applied to the CM-Sephadex column (16×2.5 cm) and left for 1 h at 4° C. The unbound protein (MRJP2 isoform X1, fraction 1) was obtained by washing the column with about 100 mL of the binding buffer. Elution of the bound protein (MRJP2, fraction 2) was achieved by a one-step gradient of about 50 mL of 0.5 M NaCl in the binding buffer. The protein content was determined in the purified fractions by UV measurement at 280 nm after dialysis for 24 h against PBS (pH 7) then freeze-dried.

### Anti-Leukemic Activities of RI Fractions

The present study evaluated the anti-leukemic effect of RJ and its isolated fractions, including carbohydrates, lipids, CPF, PF₂₅, PF₃₀, PF₄₀, PF₅₀, PF₆₀, MRJP2 and MRJP2 isoform X1 in comparison with Doxorubicin (DOX). This evaluation was done using two types of leukemia cell lines, murine myeloid (NFS-60) and human T lymphocyte (Jurkat).

Isolation of white blood cells (WBCs), The human WBCs were isolated from the blood of ten healthy volunteers (collected in heparin tubes). In brief, the blood was mixed gradually with a fresh cold lysing solution (80.2 mg % ammonium chloride, 8.4 mg % NaHCO₃, and 3.7 mg % EDTA) then centrifuged at 1650 rpm for 5 min. The pellet (WBCs) was washed twice with RPMI-1640 medium and cells were stained with trypan blue for checking the viability and counting using a phase-contrast microscope (Olympus, Tokyo, Japan). Finally, cells were cultured in RPMI-1640 medium containing 10% fetal bovine serum (FBS) and seeding as 10⁵ cells/well in 96 well cell culture plate.

Determination of the safe doses of RJ and its fractions on normal WBCs, About 100 μL of serial dilutions of each of RJ fractions and standard chemotherapy (DOX) were incubated with WBCs in a CO₂ incubator (New Brunswick Scientific, Netherlands) at 37° C., 5% CO₂, and 90% relative humidity. After 72 h, 20 μL of MTT (5 mg/mL in PBS) was added to each well and incubated for further 3 h, and then centrifuged for 10 min at 2000 rpm. One hundred microliters of DMSO was added to each well after supernatant aspiration and the absorbance was read at 570 nm using ELISA reader (BMG LabTech, Germany). Cell viability was determined and a relation between the cell viability and the studied fractions or DOX concentrations was plotted for calculating the safe concentrations (EC₁₀₀, 100% cell viability) using GraphPadInstat program.

Anti-leukemic activity of RJ and its fractions, Both leukemia cells (NFS-60 and Jurkat) were seeded in RPMI containing 10% FBS as 3000 cells/well in 96 well cell culture plate. Then serial concentrations from the safe dose of each of the tested RJ and its fractions was added and incubated for 72 h in a 5% CO₂ incubator at 37° C. The cytotoxic effect of RJ and its fractions in comparison with DOX against both leukemia cell lines were investigated using the MTT assay as described above. Then the concentration that inhibits leukemia cell growth by 50% (IG₅₀ value) was determined for the RJ and each of its fractions and used to select the effective fraction (the lowest IG₅₀ value). The morphological changes of untreated and treated leukemia cells were examined using the phase-contrast microscope.

Flow cytometric analysis of apoptosis, RJ, its effective fractions (CPF, PF₅₀, MRJP2, and MRJP2 X1), and DOX at their IG₅₀ was incubated for 72 h with each of the leukemia cell lines. After trypsinization, the untreated and treated cells were incubated with annexin V/propidium iodide (PI) for 15 min. Then cells were fixed and incubated with streptavidin-fluorescein (5 μg/mL) for 15 min. The apoptosis-dependent anti-leukemic effect was determined by quantification of annexin-stained apoptotic cells using the Fluorescein isothiocyanate (FITC) signal detector (FL1) in the flow cytometer (Partec, Germany).

Fluorescence microscope investigation of apoptotic cells, NFS-60 and Jurkat cell lines were incubated separately with the most effective RJ fractions (PF₅₀, MRJP2, and MRJP2 X1) and standard drugs (DOX) for 72 h in the CO₂ incubator. Then leukemia cell apoptosis was investigated by ethidium bromide (EB)/acridine orange (AO) double staining (100 μg/mL for each) and then visualized under the fluorescent phase contrast microscope (Olympus, Japan).

Cell cycle distribution by flow cytometry, The change in leukemia cell cycle distribution before and after treatment with IG₅₀ of the most effective anti-leukemic RJ fractions (PF₅₀, MRJP2, and MRJP2 X1) was determined by flow cytometry as described previously. Briefly, the untreated and treated leukemia cells were incubated with 5 μg/mL RNase A (Sigma, USA) then mixed with 10 μl of 1 mg/mL PI (Sigma, USA) for flow cytometry analysis at 488 nm using Cell Quist and Mod Fit softwares.

Table (1) represents the EC₁₀₀ values of RJ and its isolated fractions. Results revealed the higher values (safer) for the RJ and its isolated factions more than DOX. In addition, results elucidated the higher safety of the RJ-PFs followed by the carbohydrate fraction, RJ, then the lowest safety was the lipid fraction. The Table also showed the IG₅₀ values of RJ and its fractions against the two studied leukemia cells in comparison with DOX. Data revealed that DOX was significantly more potent than the tested fractions against the two studied leukemia cells and from these fractions, PF₅₀ was the most potent (the lowest IG₅₀).

FIG. 2 shows the morphology of the two leukemia cell lines under the phase contrast microscope after treatment with the most effective RJ-PFs and the standard chemotherapy. After 72 h incubation of the cancer cells with the different treatments, cells appeared as oval or irregular-shaped and shrinkage with condensed cytoplasm and apoptotic bodies. All of these features are the hallmarks of the apoptosis, which observed obviously with cancer cells-treated with the MRJP2 more than other treatments.

Apoptosis in the leukemia cell lines was clearly observed by annexin/PI flow cytometric analysis (FIG. 3) and the EB/AO double fluorescent staining (FIG. 4). The flow cytometric analysis showed that the highest percentage of the apoptotic cell populations was induced by MRJP2 followed by PF₅₀ then MRJP2 X1 and this apoptotic effect was nearly equipotent to DOX. While the results of EB/AO double staining clarified that the leukemia viable cell number was depleted tremendously and no necrotic cells (red enlarged nuclei) were observed with all treatments. The treatment with PF₅₀, MRJP2, and DOX increased the number of the late apoptotic cells (orange-red nuclei). However, the treatment with MRJP2 X1 elevated the number of early apoptotic cells (greenish-yellow nuclei) beside a few late apoptotic cells. In harmony with the flow cytometric results, MRJP2 showed the equipotent apoptotic effect to DOX and higher efficiency than PF₅₀ and MRJP2 X1.

FIG. 5 showed the cell cycle regulatory effect of RJ-PFs. The FIG. clarified that both types of leukemia cells were arrested and accumulated at G1 phase. After 72 h incubation of each of these cancer cells with the RJ-PFs, the arrested cell populations were significantly decreased. Interestingly, these treatments significantly delay the G0 phase and blocked the cancer cell populations in this phase. This was accompanied by a decrease the cancer cell populations in both S and G2/M phases. For all of these effects, MRJP2 was the most effective fraction with the same or higher potency than DOX. These results concomitant with the apoptotic effect of these PFs due to the strong correlation between the G0 arrest and induction of apoptosis as confirmed by many previous studies. Therefore, the anti-leukemic effect of the RJ-PFs especially, MRJP2 mediated by significant induction of G0 phase arrest followed by induction of apoptosis.

### Effect of RJ and its Fractions on the HIV-1 Reverse Transcriptase Activity

The current study evaluated the inhibitory effect of RJ and its isolated fractions (lipids, carbohydrates, CPF, PF₂₅, PF₃₀, PF₄₀, PF₅₀, PF₆₀, MRJP2, and MRJP2 isoform X1) on the HIV-1 reverse transcriptase (RT) activity. The RT assay colorimetric kit (Roche Diagnostics GmbH, Mannheim, Germany) was used. The kit principle based on the use of the template/primer hybrid poly (A)×oligo (dt)₁₅ and labeled nucleotides with digoxigenin and biotinin an optimized ratio for the synthesis of a freshly DNA molecule by RT transcriptase. The detection and quantification of the synthesized DNA follow a sandwich ELISA protocol.

For the RT inhibitory assay, the recombinant HIV-1 RT contained in the kit was prepared using autoclaved redistilled water into 10 mU/μL (2 ng/μL) final concentration. Then 20 μL (containing 1, 0.5, 0.25, 0.125, 0.0625 mg) of RJ or each isolated fractions was incubated with the same volume of the prepared enzyme (4 ng/20 μL) for 1 h at 37° C. Two controls were included, the negative control (without the enzyme) and the positive control (without the tested compounds). The enzymatic reaction was staid by adding 20 μl of the substrate mixture [template/primer hybrid (750 mA₂₆₀ nm/ml) and triphosphate (10 μM, dUTP/dTTP)] and the reaction was continued for 1 h at 37° C. Then 60 μL of the mixture was transferred into microplate (MP) modules precoated with streptavidin and post-coated with blocking reagent and incubated for another 1 h at 37° C. The MP wells were washed 5 times with the washing buffer provided by the kit, then the anti-digoxigenin-peroxidase working solution was added and followed by 1 h incubation at 37° C. The MP wells were washed again 5 times, after which the peroxidase substrate solution was added into each well and the absorbance of the produced color was measured at 405 nm using an ELISA reader (BMG LabTech, Germany). The inhibitory activity of the RJ and its fractions were calculated as percent inhibition compared to a control. Then the IC₅₀ (the concentration that inhibits 50% of the enzyme activity) was calculated for each fraction.

The results in Table 1 showed that the IC₅₀ values of all the RJ fractions are almost the same, except for the lipid fraction, which had a higher value (lower potency). When we look at the HIV-1 RT % inhibition of each of these fractions at the higher concentration used (1 mg), we can notice the most effective fractions. The PF₂₅, PF₃₀, PF₄₀, PF₆₀, and MRJP2 revealed inhibitory effect of more than 90% and they were considered the most effective RJ fractions against this enzyme. While lipid fraction was the lowest effective fraction with inhibitory effect less than 40%. Other fractions exhibited different inhibitory percentages between these two values.

### Sialidase Activity of RJ Protein Fractions

The sialidase catalytic activity of the RJ isolated PFs was evaluated by incubating different concentrations (500, 250, 125, 62.5, 31.25) of each of the RJ-PFs (CPF, PF₂₅, PF₃₀, PF₄₀, PF₅₀, PF₆₀, MRJP2 and MRJP2 isoform X1) with PBMCs or HepG2 cells at 37° C. for 2 h and 72 h. Two controls were included, each PF alone without cells and each cell alone without PF. At the end of the incubation period, the released SA concentration was quantified followed the previously published method.

Preparation of SA-attached cells, PBMCs were obtained by Ficoll-Hypaque density gradient centrifugation method as described previously. In brief, the blood samples from healthy volunteers were diluted with an equal volume of PBS, carefully layered on Ficoll-Hypaque, and centrifuged at 2000 rpm, 25° C. for 30 min. Then the undisturbed PBMCs layer (interface) was carefully transferred out, washed twice with 40 ml RPMI-1640 medium, and centrifuged at 1650 rpm for 10 min. Finally, the supernatant was removed and the cells were suspended in 5 ml of RPMI-1640 medium containing 10% FBS and counted using trypan blue stain. HepG2 cells were grown in RPMI-1640 medium (HyClone) supplemented with 10% heat-inactivated FBS.

The SA assay, SA concentration was measured by the alkali-Ehrlich method using 0.2 M borate buffer at pH 8.5. After the incubation period (2 h or 72 h), cell culture was centrifuged at 2000 rpm for 15 min and the SA content was quantified in the supernatant. To 0.5 mL of the supernatant, water (blank), or different standard concentrations (1-10 nmol/mL), 0.5 mL of the borate buffer solution was added. Then the mixture was heated at 100° C. for 45 min, cooled, treated with 3 ml of ethanol followed by 1 ml of the Ehrlich reagent and heated at 70° C. for a further 20 min. The developed violet color was read at 560 nm. The SA concentration (nmol/mL) was calculated from the standard SA calibration curve and used to calculate the sialidase activity of the RJPF as nmol/ml/min (IU). The specific activity (IU/mg protein) was calculated after determination of the protein content (mg/mL) in the supernatant using the Bradford method.

The results in FIG. 6 revealed the ability of all the studied RJ-PFs except PF₂₅ and PF₆₀ to release SA from the surface of PBMCs and HepG2 cells (i.e having sialidase activity) and this ability was time (FIG. 6E) and concentration (FIG. 6A-D)-dependent. The most potent enzymatic activity was observed for the PF₅₀ more than other RJ-PFs and its purified proteins (MRJP2 and MRJP2 X1) separately. This effect clarified the synergistic catalytic activity of MRJPs in a combined form (PF₅₀). The sialidase activity of the RJ-PFs, particularly PF₅₀ has a crucial role in the prevention of many viruses, bacteria, and toxins entry into their host cells. Therefore, by cleaving SAs from the surface of the host cells, their receptors will be inactivated and thereby potentially renders the host cells resistant to this target infection.

### Statistics

Data were expressed as mean±SE and were analyzed by SPSS version 16. The mean values were compared using one-way analysis of variance (ANOVA) by Duncan's test and significance was determined at P<0.05. IC₅₀ and EC₁₀₀ values were calculated by the GraphPadInstat software version 3.

### A BRIEF DESCRIPTION OF THE DRAWING

FIG. 1: Novel activities of Apis mellifera royal jelly proteins.

FIG. 2: Morphological changes in the murine myeloid (NFS-60) and human T lymphocyte (Jurkat) leukemia cell lines after the treatment with royal jelly (RJ) and its protein fractions (PFs) in comparison with the doxorubicin (DOX) chemotherapeutic drug as observed under the inverted microscope. CPF; crude protein fraction, MRJP; major royal jelly protein.

FIG. 3: Flow cytometric analysis using annexin V/propidium iodide (PI) double staining for detection of the apoptotic leukemia cells before and after the treatment with royal jelly (RJ) and its protein fractions (PFs) in comparison with the doxorubicin (DOX) chemotherapeutic drug. (A) Annexin V/PI flow charts for the control and treated-murine myeloid (NFS-60) and human T lymphocyte (Jurkat) leukemia cell lines. (B) Quantification of the % apoptotic cells in the control and treated-leukemia cells. CPF; crude protein fraction, MRJP; major royal jelly protein. Values are presented as mean±SE (n=3) and different letters specify the significance at P<0.05.

FIG. 4: Acridine orange/ethidium bromide nuclear double staining of the apoptotic cell populations in the murine myeloid (NFS-60) and human T lymphocyte (Jurkat) leukemia cells before and after the treatment with the effective royal jelly (RJ) protein factions (PFs) in comparison with the doxorubicin (DOX) chemotherapeutic drug. MRJP; major royal jelly protein, VC; viable cells, EA, LA; early and late apoptotic cells, respectively.

FIG. 5: Cell cycle distribution of murine myeloid (NFS-60) and human T lymphocyte (Jurkat) leukemia cells before and after the treatment with the effective royal jelly (RJ) protein factions (PFs) in comparison with the doxorubicin (DOX) chemotherapeutic drug. (A) Flow cytometric images showed G2/M phase arrest (B, C) Quantification of the percentage of cells in the cell cycle phases (G0, G1, S, and G2/M). MRJP; major royal jelly protein. Values are presented as mean±SE (n=3) and different letters specify the significance at P<0.05.

FIG. 6: Sialidase activity of royal jelly (RJ) protein fractions (PFs). (A, C) After 2 h incubation with peripheral blood mononuclear cells (PBMCs) and HepG2 cell, respectively (B, D) After 72 h incubation with PBMCs and HepG2 cell, respectively. (E) Time-dependent sialidase activity of RJ-PFs at the concentration of 500 μg/mL. CPF; crude protein fraction, MRJP; major royal jelly protein.

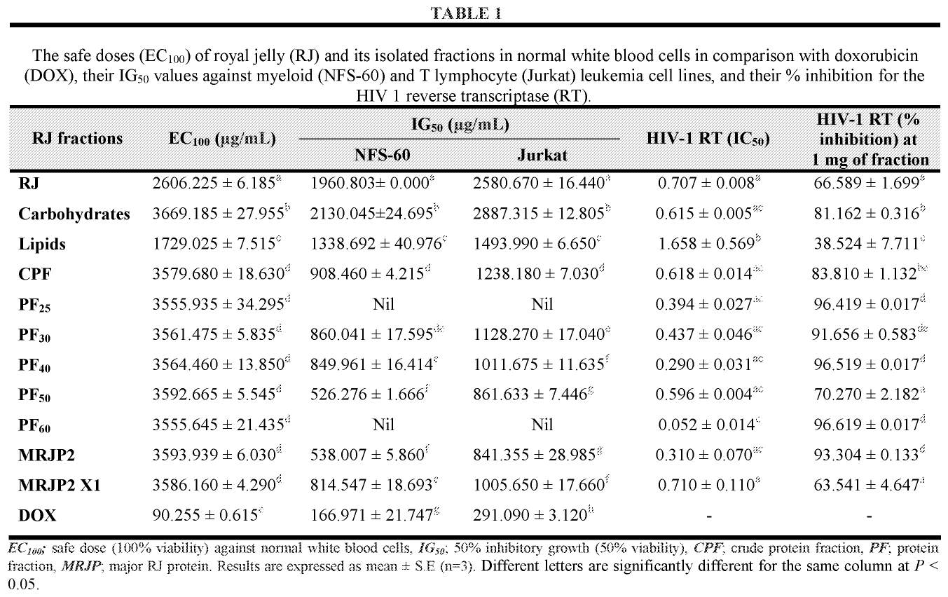

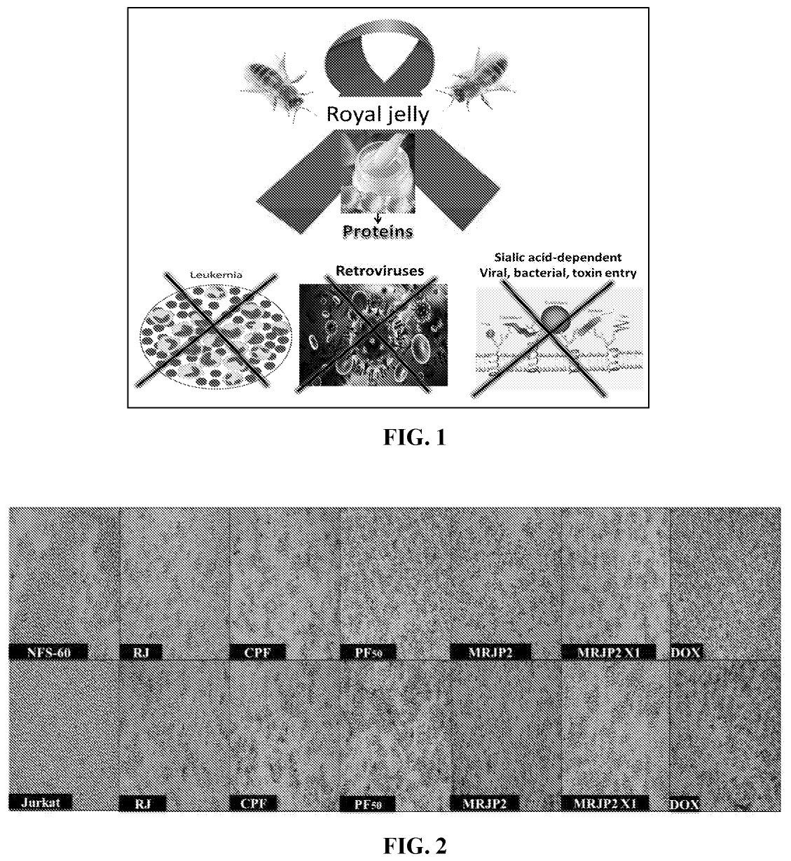

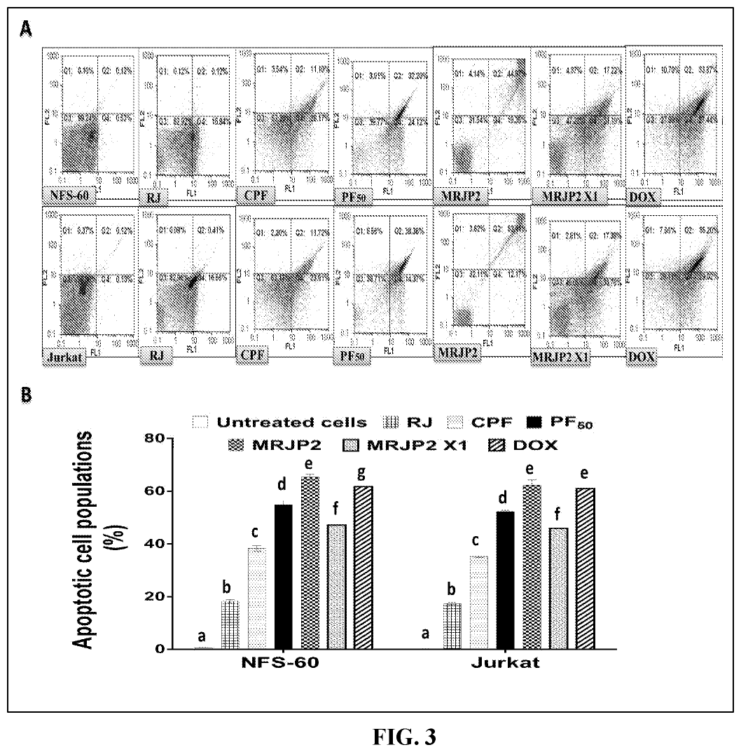

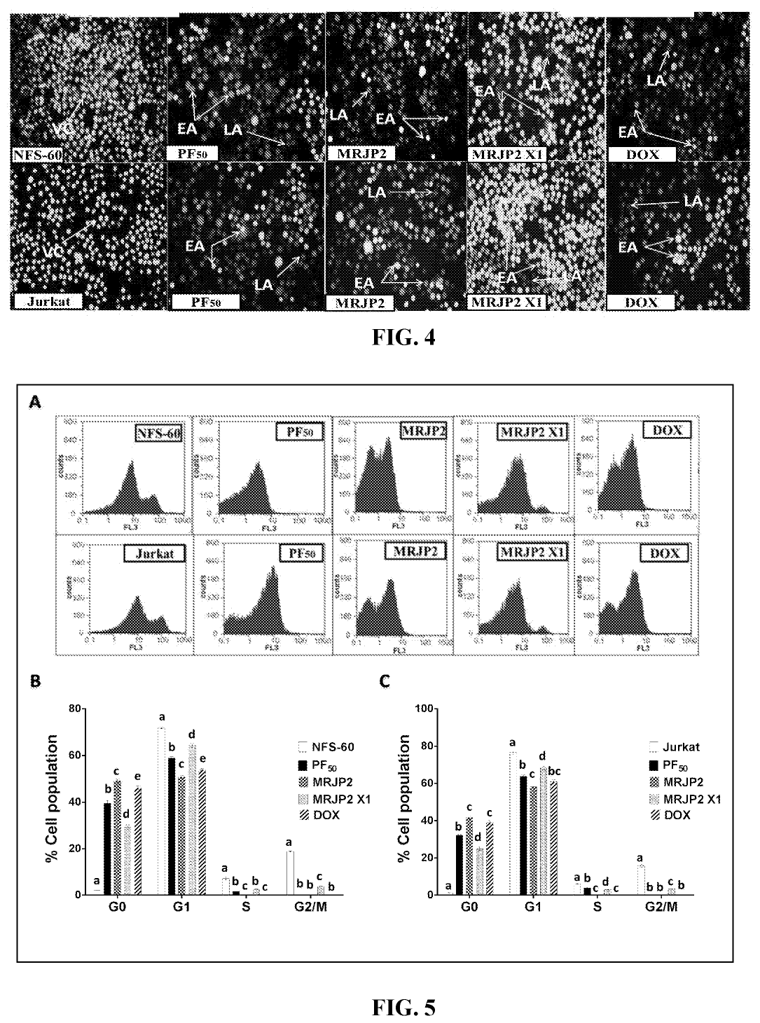

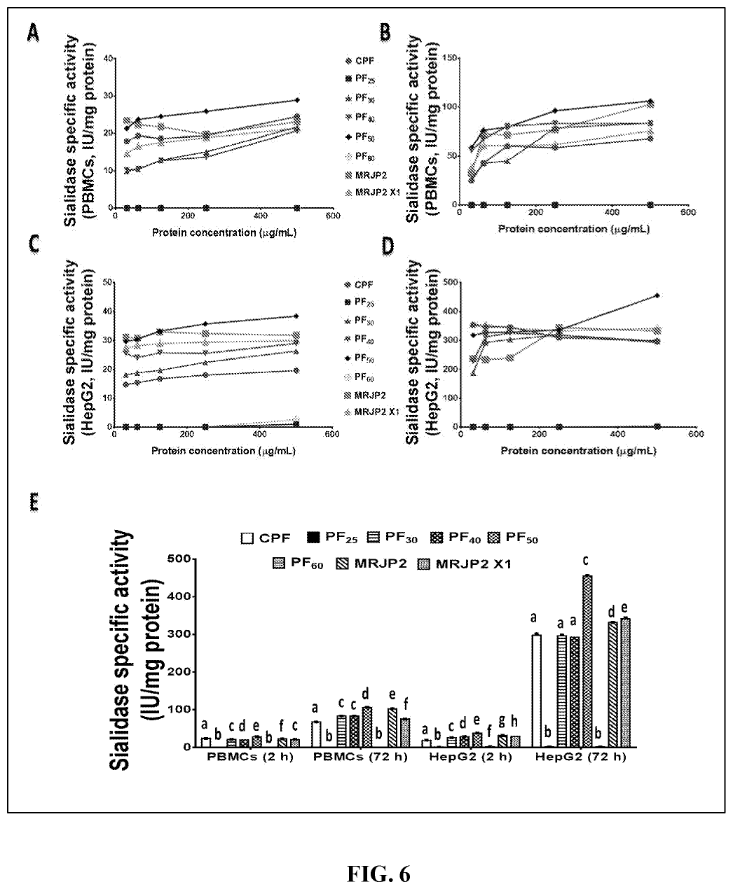

1. A method of inhibiting myeloid and lymphoid leukemia cell growth and HIV-1 reverse transcriptase, RT, activity, the method comprising administering to the cells different protein fractions isolated from Apis mellifera royal jelly, RJ, named as protein fraction 25, PF₂₅, 30, PF₃₀, 40, PF₄₀, 50, PF₅₀, 60, PF₆₀, major royal jelly protein 2, MRJP2, and MRJP2 isoform XI, resulting in inhibitory effects of different potency for myeloid and lymphoid leukemia cell growth and HIV-1 reverse transcriptase, RT, activity, and where some of the protein fractions having sialidase catalytic activity.

2. A method of inhibiting the myeloid and lymphoid leukemia cell growth according to claim 1, comprising the use of PF₃₀ or one of its proteins.

3. A method of inhibiting the myeloid and lymphoid leukemia cell growth according to claim 1, comprising the use of PF₄₀ or one of its proteins.

4. A method of inhibiting the myeloid and lymphoid leukemia cell growth according to claim 1, comprising the use of PF₅₀

5. A method of inhibiting the myeloid and lymphoid leukemia cell growth according to claim 1, comprising the use of MRJP2.

6. A method of inhibiting the myeloid and lymphoid leukemia cell growth according to claim 1, comprising the use of MRJP2 isoform XI.

7. A method of inhibiting the HIV-1 RT activity according to claim 1, comprising the use of PF₂₅ or one of its proteins.

8. A method of inhibiting the HIV-1 RT activity according to claim 1, comprising the use of PF₃₀ or one of its proteins.

9. A method of inhibiting the HIV-1 RT activity according to claim 1, comprising the use of PF₄₀ or one of its proteins.

10. A method of inhibiting the HIV-1 RT activity according to claim 1, comprising the use of PF₆₀ or one of its proteins.

11. A method of inhibiting the HIV-1 RT activity according to claim 1, comprising the use of MRJP2.

12. Based on claims 7-11, PF₂₅, PF₃₀, PF₄₀, PF₆₀, and MRJP2 can prohibit the HIV replication and similarly other retroviruses such as Oncoviruses, Spumavirus, and many others in addition to HBV.

13. According to claim 1, the PF₃o or one of its proteins able to release sialic acids (SAs) from the cellular surface i.e. they have sialidase activity.

14. According to claim 1, the PF₄₀ or one of its proteins able to release sialic acids (SAs) from the cellular surface i.e. they have sialidase activity.

15. According to claim 1, the PF₅₀ able to release sialic acids (SAs) from the cellular surface i.e. it has sialidase activity.

16. According to claim 1, the MRJP2 able to release sialic acids (SAs) from the cellular surface i.e. it has sialidase activity.

17. According to claim 1, the MRJP2 isoform XI able to release sialic acids (SAs) from the cellular surface i.e. it has sialidase activity.

18. Based on claims 13-17, PF₃₀, PF₄₀, and PF₅₀ or one of their proteins in addition to MRJP2 and MRJP2 isoform XI able to prevent the infection with viruses that their entry to the host cells depends on the SA receptor such as Influenza virus, Isavirus Coronaviruses [including severe acute respiratory syndrome coronavirus 2], Adenoviruses, Rotaviruses, and many others.

19. Based on claims 13-17, PF₃₀, PF₄₀, and PF₅₀ or one of their proteins in addition to MRJP2 and MRJP2 isoform XI able to prevent the infection with some types of bacteria and bacterial toxins that depend on the presence of SA receptor on the host cells to begin their infection. These include Helicobacter pylori, Streptococcus pneumoniae, Vibrio cholera toxin, Clostridium tetani toxin, and others.
